(19) United States
(12) Patent Application Publication
(10) Pub. No.: US 2004/0186089 A1
(43) Pub. Date: Sep. 23, 2004

(54) Compounds useful in the treatment of anthrax

(76) Inventor: Edward M. Scolnick, Wynnewood, PA (US)

Correspondence Address: MERCK AND CO INC, P O BOX 2000, RAHWAY, NJ, 070650907

(21) Appl. No.: 10/491,235

(22) PCT Filed: Nov. 4, 2002
(86) PCT No.: PCT/US02/35342

(30) Foreign Application Priority Data
Nov. 8, 2001 (US) 60337507

(51) Int. Cl. A61K 31/41

(52) U.S. Cl. 514/210.13

(57) ABSTRACT

This invention relates to a method of inhibiting growth of Bacillus anthracis, Bacillus cereus and/or Bacillus thuringiensis bacterial strains in a mammal comprising administration to a patient in need thereof a therapeutically effective amount of a carbapenem compound. In particular, this invention relates to a method of inhibiting growth of Bacillus anthracis, Bacillus cereus and/or Bacillus thuringiensis bacterial strains in a mammal using compounds having the structural formula (I) or a pharmaceutically acceptable salt, enantiomer, diastereomer or in vivo hydrolysable ester or mixture thereof. This invention further relates to the use of carbapenem compounds of formula I in the treatment of anthrax and other conditions which are related to an anthrax infection.

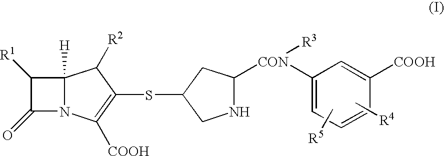

### BACKGROUND OF THE INVENTION

Bacillus anthracis is a spore forming gram-positive bacillus, which is the etiologic agent of anthrax. Anthrax is a disease that can be found globally in temperate zones (e.g. South and Central America, South and East Europe, Asia, Africa, Middle East, and Caribbean) and is transmissible to humans through handling or consumption of contaminated animal products (e.g. eating undercooked meat from infected animals). Wildlife mammals such as deer, wildebeest, elephants, and domesticated livestock, such as goats, sheep, cattle, horses, and swine are at high risk for contracting the disease. Contraction generally occurs from grazing on contaminated land, eating contaminated feed or drinking from contaminated water holes. Bacillus anthracis spores can remain viable in soil for many years. See Helgason et al., Applied and Environmental Microbiology 2000 66(6) pgs. 2627-2630; Wber et al., Antimicrob Agents and Chemotherapy 1988 32(5): 642-645; and Doganay et al., Scand. J. Inf. Dis. 1991 23:333-335 for further discussion of Bacillus anthracis.

In humans three forms of anthrax can occur, cutaneous, gastro-intestinal and inhalational. With the cutaneous form, infections occur when the bacterium or spore enters a cut or abrasion on the skin. See Synder, J. W., Shapiro, D. S., Gilchrist, M. J. R., et al., “Basic Diagnostic Testing Protocols for Level A Laboratories Nor The Presumptive Indentification of Bacillus anthracis)” at website: www.ban.asm.la.cp.102401f, Oct. 24, 2001, pgs. 1-20 and Dixon, et al., NEJM 341:815-826 Sep. 9, 1999 Number 11. Symptoms of the skin infection are generally raised itchy bumps or bump that resembles an insect bite. Within one to two days, the bumps or bump develops into a fluid-filled vesicle, which ruptures to form a painless ulcer with a characteristic black necrotic (dying) area in the center. If left untreated, death can result, however, deaths are rare if appropriate antibiotic therapy is administered.

Gastrointestinal anthrax generally occurs from the consumption of meat contaminated with the bacterium, which results in an acute inflammation of the intestinal tract. Signs of nausea, loss of appetite, vomiting, fever, along with abdominal pain, vomiting of blood and severe diarrhea are indicative of gastrointestinal anthrax. The mortality rate for this form of human anthrax is estimated at 25%-60%.

Inhalation anthrax is most likely the result of intentional aerosol release of Bacillus anthracis, such as an act of bioterrorism. This form of human anthrax infection commonly has an incubation period of one to six days, with fever, malaise, fatigue, a nonproductive cough and/or mild chest discomfort sometimes being the initial signals. These initial symptoms are often followed by a short period of improvement, followed by the abrupt development of sever respiratory distress with labored breathing, perspiration and bluish skin color. Death usually occurs within 24-36 hours after the onset of respiratory distress despite aggressive treatment.

Most Bacillus anthracis strains are sensitive to a broad range of antibiotics. The commonly prescribed therapies today are ciprofloxacin, penicillin, or doxycycline. However, the efficacy and side effect profiles of these agents are not ideal. Therefore, there still exist the need for new and effective therapies with improved efficacy and little or no side effects.

### SUMMARY OF THE INVENTION

This invention relates to a method of inhibiting growth of Bacillus anthracis bacterial strains or homologues thereof in a mammal comprising administration to a patient in need thereof, a therapeutically effective amount of a carbapenem compound. In particular, this invention relates to a method of inhibiting growth of Bacillus anthracis, Bacillus cereus and/or Bacillus thuringiensis bacterial strains in a mammal using compounds having the structural formula I:

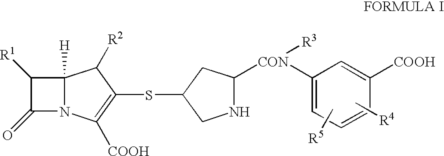

or a pharmaceutically acceptable salt, enantiomer, diastereomer or in vivo hydrolysable ester or mixture thereof:

wherein,

R¹ represents 1-hydroxyethyl, 1-fluoroethyl or hydroxymethyl;

R² and R³ independently represent hydrogen or C₁₋₄ alkyl;

R⁴ and R⁵ are the same or different and are selected from hydrogen, halo, cyano, C₁₋₄ alkyl, nitro, hydroxy, carboxy, C₁₋₄ alkoxy, C₁₋₄ alkoxycarbonyl, aminosulphonyl, C₁₋₄ alkylaminosulphonyl, di-C₁₋₄ alkylaminosulphonyl, carbamoyl, C₁₋₄ alkylcarbamoyl, di-C₁₋₄ alkylcarbamoyl, trifluoromethyl, sulphonic acid, amino, C₁₋₄ alkylamino, di-C₁₋₄ alkylamino, C₁₋₄ alkanoylamino, C₁₋₄ alkanoyl(N—C₁₋₄ alkyl)amino, C₁₋₄ alkanesulphonamido and C₁₋₄ alkylS(O)ₙ— wherein n is 0-2:

With the proviso that there is no hydroxy or carboxy substituent in a position ortho to the link to —NR³—.

This invention further relates to the use of carbapenem compounds of formula I in the treatment of anthrax and other conditions which are related to an anthrax infection.

This and other aspects of the invention will be realized upon inspection of the invention as a whole.

### DETAILED DESCRIPTION OF THE INVENTION

The present invention is directed to a method for treating anthrax by administration, preferably intravenous or intra-muscular, of a composition containing a carbapenem of formula I and a pharmaceutically acceptable carrier.

The invention is described herein in detail using the terms defined below unless otherwise specified.

The term “alkyl” refers to a monovalent alkane (hydrocarbon) derived radical containing from 1 to 10 carbon atoms unless otherwise defined. It may be straight, branched or cyclic. Preferred alkyl groups include methyl, ethyl, propyl, isopropyl, butyl, t-butyl, cyclopentyl and cyclohexyl. When the alkyl group is said to be substituted with an alkyl group, this is used interchangeably with “branched alkyl group”.

Cycloalkyl is a specie of alkyl containing from 3 to 15 carbon atoms, without alternating or resonating double bonds between carbon atoms. It may contain from 1 to 4 rings which are fused. Example of cycloalkyl groups are cyclopropyl, cyclobutyl, cyclopentyl, cyclohexyl, and cycloheptyl.

Alkoxy refers to C₁-C₆ alkyl-O—, with the alkyl group optionally substituted as described herein. Examples of alkoxy groups are methoxy, ethoxy, propoxy, butoxy and isomeric groups thereof.

Halo is short for halogen and refers to chloride, fluoride, bromide and iodide.

Preferably R¹ is 1-hydroxyethyl, R² is hyrogen or methyl, R³ is hydrogen, and R⁴ and R⁵ are the same or different and are selected from hydrogen, fluoro, chloro, hydroxy, carboxy, cyano, nitro, methyl, ethyll, methoxy, ethoxy, methoxycarbonyl, carbamoyl, methylcarbamoyl, dimethylcarbamoyl, trifluoromethyl, sulphonic acid, methylsulphinyl, methylsulphonyl, methanesulphonamido or acetamido.

R⁴ and R⁵ may both be other than hydrogen but, in general, it is particularly preferred that at least one of R⁴ and R⁵ is hydrogen.

Particularly preferred compounds used in this invention are those in which R⁴ is hydrogen, carboxy, fluoro, chloro, methyl, methoxy, cyano, sulphonic acid or methoxycarbonyl and R⁵ is hydrogen.

Suitable pharmaceutically acceptable salts of the compounds used in this invention include acid addition salts such as hydrochloride, hydrobromide, citrate, maleate and salts formed with phosphoric and sulphuric acid. In another aspect suitable salts are base salts such as an alkali metal salt for example sodium or potassium, an alkaline earth metal salt for example calcium or magnesium, an organic amine salt for example triethylamine, morpholine, N-methylpiperidine, N-ethylpiperidine, procaine, dibenzylamine, N,N-dibenzylethylamine or amino acids for example lysine. Preferred pharmaceutically acceptable salts are sodium and potassium salts.

In vivo hydrolysable esters are those pharmaceutically acceptable esters that hydrolyze in the human body to produce the parent compound. Such esters can be identified by administering, e.g. intravenously to a test animal, the compound under test and subsequently examining the test animal's body fluids. Suitable in vivo hydrolysable esters for carboxy include C₁₋₆alkoxymethyl esters for example methoxymethyl, C₁₋₆ alkanolyloxymethyl esters for example pivaloyloxymethyl, phthalidyl esters and the additional esters disclosed in U.S. Pat. No. 5,478,820, which is herein incorporated by reference in its entirety.

For purposes of this invention, Bacillus anthracis homologs include Bacillus cereus and Bacillus thuringiensis.

Preferred compounds used in this invention are:

(1R,5S,6S,8R,2′S,4′S)-2-(2-(3-carboxy-5-hydroxyphenyl-carbamoyl)pyrrolidin-4-ylthio)-6-(1-hydroxyethyl)-1-methylcarbapenem-3-carboxylic acid;

(1R,5S,6S,8R,2′S,4′S)-2-(2-(3-carboxy-4-chlorophenylcarbamoyl)pyrrolidin-4-ylthio)-6-(1-hydroxyethyl)-1-methylcarbapenem-3-carboxylic acid;

(1R,5S,6S,8R,2′S,4′S)-2-(2-(3-carboxy-6-chlorophenylcarbamoyl)pyrrolidin-4-ylthio)-6-(1-hydroxyethyl)-1-methylcarbapenem-3-carboxylic acid;

(1R,5S,6S,8R,2′S,4′S)-2-(2-(3-carboxyphenylcarbamoyl)pyrrolidin-4-ylthio)-6-(1-hydroxyethyl)-1-methylcarbapenem-3-carboxylic acid;

(1R,5S,6S,8R,2′S,4′S)-2-(2-(3-carboxy-6-methanesulphonylphenylcarbamoyl)pyrrolidin-4-ylthio)-6-(1-hydroxyethyl)-1-methylcarbapenem-3-carboxylic acid;

(1R,5S,6S,8R,2′S,4′S)-2-(2-(3-carboxy-4-fluorophenylcarbamoyl)pyrrolidin-4-ylthio)-6-(1-hydroxyethyl)-1-methylcarbapenem-3-carboxylic acid;

(1R,5S,6S,8R,2′S,4′S)-2-(2-(3-carboxy-6-fluorophenylcarbamoyl)pyrrolidin-4-ylthio)-6-(1-hydroxyethyl)-1-methylcarbapenem-3-carboxylic acid;

(1R,5S,6S,8R,2′S,4′S)-2-(2-(3-carboxy-2,4-difluorophenylcarbamoyl)pyrrolidin-4-ylthio)-6-(1-hydroxyethyl)-1-methylcarbapenem-3-carboxylic acid;

(1R,5S,6S,8R,2′S,4′S)-2-(2-(3,4-dicarboxyphenylcarbamoyl)pyrrolidin-4-ylthio)-6-(1-hydroxyethyl)-1-methylcarbapenem-3-carboxylic acid;

(1R,5S,6S,8R,2′S,4′S)-2-(2-(3-carboxy-4-hydroxyphenylcarbamoyl)pyrrolidin-4-ylthio)-6-(1-hydroxyethyl)-1-methylcarbapenem-3-carboxylic acid;

(1R,5S,6S,8R,2′S,4′S)-2-(2-(3,5-dicarboxyphenylcarbamoyl)pyrrolidin-4-ylthio)-6-(1-hydroxyethyl)-1-methylcarbapenem-3-carboxylic acid;

(1R,5S,6S,8R,2′S,4′S)-2-(2-(2-carbamoyl-3-carboxyphenylcarbamoyl)pyrrolidin-4-ylthio)-6-(1-hydroxyethyl)-1-methylcarbapenem-3-carboxylic acid;

(1R,5S,6S,8R,2′S,4′S)-2-(2-(3-carboxy-4-carbamoylphenylcarbamoyl)pyrrolidin-4-ylthio)-6-(1-hydroxyethyl)-1-methylcarbapenem-3-carboxylic acid;

(1R,5S,6S,8R,2′S,4′S)-2-(2-(3-carboxy-5-carbamoylphenylcarbamoyl)pyrrolidin-4-ylthio)-6-(1-hydroxyethyl)-1-methylcarbapenem-3-carboxylic acid;

(1R,5S,6S,8R,2′S,4′S)-2-(2-(3-carboxy-5-acetamidophenylcarbamoyl)pyrrolidin-4-ylthio)-6-(1-hydroxyethyl)-1-methylcarbapenem-3-carboxylic acid;

(1R,5S,6S,8R,2′S,4′S)-2-(2-(3-carboxy-4-acetamidophenylcarbamoyl)pyrrolidin-4-ylthio)-6-(1-hydroxyethyl)-1-methylcarbapenem-3-carboxylic acid;

(1R,5S,6S,8R,2′S,4′S)-2-(2-(3-carboxy-5-methylsulphonamidophenylcarbamoyl)pyrrolidin-4-ylthio)-6-(1-hydroxyethyl)-1-methylcarbapenem-3-carboxylic acid;

(1R,5S,6S,8R,2′S,4′S)-2-(2-(3-carboxy-5-sulphophenylcarbamoyl)pyrrolidin-4-ylthio)-6-(1-hydroxyethyl)-1-methylcarbapenem-3-carboxylic acid;

(1R,5S,6S,8R,2′S,4′S)-2-(2-(3-carboxy-6-carbamoylphenylcarbamoyl)pyrrolidin-4-ylthio)-6-(1-hydroxyethyl)-1-methylcarbapenem-3-carboxylic acid;

(1R,5S,6S,8R,2′S,4′S)-2-(2-(3-carboxy-2-dimethylaminocarbonylphenylcarbamoyl)-pyrrolidinylthio)-6-(1-hydroxyethyl)-1-methylcarbapenem-3-carboxylic acid;

(1R,5S,6S,8R,2′S,4′S)-2-(2-(3-carboxyphenylcarbamoyl)pyrrolidin-4-ylthio)-6-(1-hydroxyethyl)-1-methylcarbapenem-3-carboxylic acid;

(1R,5S,6S,8R,2′S,4′S)-2-(2-(3-carboxymethylphenylcarbamoyl)pyrrolidin-4-ylthio)-6-(1-hydroxyethyl)-1-methylcarbapenem-3-carboxylic acid;

(1R,5S,6S,8R,2′S,4′S)-2-(2-(3-carboxy-5-methylphenylcarbamoyl)pyrrolidin-4-ylthio)-6-(1-hydroxyethyl)-1-methylcarbapenem-3-carboxylic acid;

(1R,5S,6S,8R,2′S,4′S)-2-(2-(3-carboxy-6-methylphenylcarbamoyl)pyrrolidin-4-ylthio)-6-(1-hydroxyethyl)-1-methylcarbapenem-3-carboxylic acid;

(1R,5S,6S,8R,2′S,4′S)-2-(2-(3-carboxy-2-methoxyphenylcarbamoyl)pyrrolidin-4-ylthio)-6-(1-hydroxyethyl)-1-methylcarbapenem-3-carboxylic acid;

(1R,5S,6S,8R,2′S,4′S)-2-(2-(3-carboxy-4-methoxyphenylcarbamoyl)pyrrolidin-4-ylthio)-6-(1-hydroxyethyl)-1-methylcarbapenem-3-carboxylic acid;

(1R,5S,6S,8R,2′S,4′S)-2-(2-(3-carboxy-5-methoxyphenylcarbamoyl)pyrrolidin-4-ylthio)-6-(1-hydroxyethyl)-1-methylcarbapenem-3-carboxylic acid;

(1R,5S,6S,8R,2′S,4′S)-2-(2-(3-carboxy-6-methoxyphenylcarbamoyl)pyrrolidin-4-ylthio)-6-(1-hydroxyethyl)-1-methylcarbapenem-3-carboxylic acid;

(1R,5S,6S,8R,2′S,4′S)-2-(2-(3-carboxy-4,6-dimethoxyphenylcarbamoyl)pyrrolidin-4-ylthio)-6-(1-hydroxyethyl)-1-methylcarbapenem-3-carboxylic acid;

(1R,5S,6S,8R,2′S,4′S)-2-(2-(3-carboxy-5-methoxycarbonylphenylcarbamoyl)pyrrolidinylthio)-6-(1-hydroxyethyl)-1-methylcarbapenem-3-carboxylic acid;

(1R,5S,6S,8R,2′S,4′S)-2-(2-(3-carboxy-5-cyanophenylcarbamoyl)pyrrolidin-4-ylthio)-6-(1-hydroxyethyl)-1-methylcarbapenem-3-carboxylic acid;

(1R,5S,6S,8R,2′S,4′S)-2-(2-(3-carboxy-5-trifluoromethylphenylcarbamoyl)pyrrolidin-4-ylthio)-6-(1-hydroxyethyl)-1-methylcarbapenem-3-carboxylic acid;

(1R,5S,6S,8R,2′S,4′S)-2-(2-(3-carboxy-4,6-difluorophenylcarbamoyl)pyrrolidin-4-ylthio)-6-(1-hydroxyethyl)-1-methylcarbapenem-3-carboxylic acid;

(1R,5S,6S,8R,2′S,4′S)-2-(2-(3-carboxy-6-methylsulphinylphenylcarbamoyl)pyrrolidin-4-ylthio)-6-(1-hydroxyethyl)-1-methylcarbapenem-3-carboxylic acid;

(1R,5S,6S,8R,2′S,4′S)-2-(2-(3-carboxy-5-methylsulphonylphenylcarbamoyl)pyrrolidin-4-ylthio)-6-(1-hydroxyethyl)-1-methylcarbapenem-3-carboxylic acid;

(1R,5S,6S,8R,2′S,4′S)-2-(2-(3-carboxy-5-fluorophenylcarbamoyl)pyrrolidin-4-ylthio)-6-(1-hydroxyethyl)-1-methylcarbapenem-3-carboxylic acid;

(1R,5S,6S,8R,2′S,4′S)-2-(2-(3-carboxy-6-cyanophenylcarbamoyl)pyrrolidin-4-ylthio)-6-(1-hydroxyethyl)-1-methylcarbapenem-3-carboxylic acid;

(1R,5S,6S,8R,2′S,4′S)-2-(2-(3-carboxy-N′-methylphenylcarbamoyl)pyrrolidin-4-ylthio)-6-(1-hydroxyethyl)-1-methylcarbapenem-3-carboxylic acid; and

pharmaceutically acceptable salts, enantiomers, diastereomers or in vivo hydrolysable esters or mixtures thereof.

More preferred compounds used in this invention are:

(1R,5S,6S,8R,2′S,4′S)-2-(2-(3-carboxy-5-methylphenylcarbamoyl)pyrrolidin-4-ylthio)-6-(1-hydroxyethyl)-1-methylcarbapenem-3-carboxylic acid;

(1R,5S,6S,8R,2′S,4′S)-2-(2-(3-carboxy-5-methoxyphenylcarbamoyl)pyrrolidin-4-ylthio)-6-(1-hydroxyethyl)-1-methylcarbapenem-3-carboxylic acid;

(1R,5S,6S,8R,2′S,4′S)-2-(2-(3-carboxy-6-methoxyphenylcarbamoyl)pyrrolidin-4-ylthio)-6-(1-hydroxyethyl)-1-methylcarbapenem-3-carboxylic acid;

(1R,5S,6S,8R,2′S,4′S)-2-(2-(3-carboxy-5-methoxycarbonylphenylcarbamoyl)pyrrolidinylthio)-6-(1-hydroxyethyl)-1-methylcarbapenem-3-carboxylic acid;

(1R,5S,6S,8R,2′S,4′S)-2-(2-(3-carboxy-5-cyanophenylcarbamoyl)pyrrolidin-4-ylthio)-6-(1-hydroxyethyl)-1-methylcarbapenem-3-carboxylic acid;

(1R,5S,6S,8R,2′S,4′S)-2-(2-(3-carboxy-6-chlorophenylcarbamoyl)pyrrolidin-4-ylthio)-6-(1-hydroxyethyl)-1-methylcarbapenem-3-carboxylic acid;

(1R,5S,6S,8R,2′S,4′S)-2-(2-(3-carboxyphenylcarbamoyl)pyrrolidin-4-ylthio)-6-(1-hydroxyethyl)-1-methylcarbapenem-3-carboxylic acid;

(1R,5S,6S,8R,2′S,4′S)-2-(2-(3-carboxy-4-fluorophenylcarbamoyl)pyrrolidin-4-ylthio)-6-(1-hydroxyethyl)-1-methylcarbapenem-3-carboxylic acid;

(1R,5S,6S,8R,2′S,4′S)-2-(2-(3-carboxy-6-fluorophenylcarbamoyl)pyrrolidin-4-ylthio)-6-(1-hydroxyethyl)-1-methylcarbapenem-3-carboxylic acid;

(1R,5S,6S,8R,2′S,4′S)-2-(2-(3,4-carboxyphenylcarbamoyl)pyrrolidin-4-ylthio)-6-(1-hydroxyethyl)-1-methylcarbapenem-3-carboxylic acid;

(1R,5S,6S,8R,2′S,4′S)-2-(2-(3,5-dicarboxyphenylcarbamoyl)pyrrolidin-4-ylthio)-6-(1-hydroxyethyl)-1-methylcarbapenem-3-carboxylic acid;

(1R,5S,6S,8R,2′S,4′S)-2-(2-(3-carboxy-5-sulphophenylcarbamoyl)pyrrolidin-4-ylthio)-6-(1-hydroxyethyl)-1-methylcarbapenem-3-carboxylic acid;

and pharmaceutically acceptable salts, enantiomers, diastereomers or in vivo hydrolysable esters or mixtures thereof.

A most preferred compound of formula I is (1R,5S,6S,8R,2′S,4′S)-2-(2-(3-carboxyphenylcarbamoyl)pyrrolidin-4-ylthio)-6-(1-hydroxyethyl)-1-methylcarbapenem-3-carboxylic acid in the form of a monosodium salt, disodium salt or mixture thereof, with the most preferred being the monosodium salt, known as ertapenem. The in vitro activity of ertapenem against certain clinical isolates has been discussed. See Fuchs et al., Antimic Agents and Chemotherapy 2001; 45(6) 1915-1918 and Livermore et al., Antimic Agents and Chemotherapy 2001; 45(6) 1860-1967. However, the susceptability of Bacillus Anthracis and homologs thereof to ertapenem has only been recently appreciated.

This invention is also concerned with a method of inhibiting the growth of Bacillus Anthracis, Bacillus cereus and/or Bacillus thuringiensis bacterial strains or treating anthrax by administering to a patient in need thereof one of the compounds of formula I alone or in combination with one or more known drugs selected from other clinically useful antibacterial agents (for example other beta-lactams or aminoglycosides), inhibitors of beta-lactamase, renal tubular blocking agents (e.g. probenecid) and inhibitors of metabolising enzymes (for example inhibitors of dehydropeptidases, for example Z-2-acylamino-3-substituted propenoates such as cilastatin) and N-acylated amino acids (for example see EP-A-178911) which reduce adverse effects on the kidney. Examples of drugs that can be combined with the compounds of formula I are imipenem, meropenem, vancomycin, cilastatin, cefoxitin, penicillin, clavulanic acid, probenecid, tetracycline, ciprofloxacin, norfloxacin or a mixture thereof. Carbapenems such as imipenem and meropenem have been tested against several clinical isolates (see Belobraybic et al., Exp. Clin. Pharmacol. 1986; November: 8(11) 675-678; and Kayser et al., J. Antimicrob Chemother 1989 September; 24 Suppl A: 101-112). The latest (Morbidity and Mortality Weekly Report) Oct. 26, 2001 Vol 50 No.42 p.909-919 cites some of the antibiotics that can work in anthrax including imipenem.

Thus, another aspect of this invention is concerned with a method for treating anthrax by administering to a patient in need thereof imipenem (N-formimidoyl thienamycin) formulated alone or in combination with inhibitors of dehydropeptidases, such as cilastatin. It is preferred to treat anthrax using the combination of imipenem and cilastatin, which is marketed as PRIMAXIN®. Imipenem ([5R-[5α,6α(R*)]]-6-(1-hydroxyethyl)-3-[[2-[(iminomethyl)amino]ethyl]thio]-7-oxo-1-azabicyclo[3.2.0]hept-2-ene-2-carboxylic acid monohydrate), cilastatin ([R-[R*,S*-(Z)]]-7-[(2-amino-2-carboxyethyl)thio]-2-[[(2,2-dimethylcyclopropyl)carbonyl]amino]-2-heptenoic acid, monosodium salt) and PRIMAXIN®, formulations and methods of making the same are disclosed in U.S. Pat. Nos. 4,194,047; 5,147,868 and 4,539,208, respectively, all of which are incorporated by reference in their entirety.

In order to use a compound of formula I or a pharmaceutically acceptable salt, enantiomer, diastereomer or in vivo hydrolysable ester or mixture thereof for the therapeutic treatment of mammals, including humans, in particular in treating anthrax, it is normally formulated in accordance with standard pharmaceutical practice as a pharmaceutical composition.

The compounds used in the instant invention can be administered in a therapeutically effective amount intravaneously, subcutaneously, intramuscularly or any other method known to those skilled in the art (e.g., rectal, oral, parenteral). A suitable pharmaceutical composition used in this invention is one, which is made for sterile injection containing between 1 and 50% w/w of the compounds used in this invention.

Suitable subjects for the administration of the formulation of the present invention include primates, man and other animals, particularly man and domesticated animals such as cats, rabbits and dogs.

The compounds of formula I and processes for making the same are disclosed in U.S. Pat. No. 5,478,820, the contents of which are all incorporated by reference in their entirety.

The following non-limiting examples, given by way of illustration, is demonstrative of the present invention, that the compounds used in this invention are useful for treating anthrax.

To determine the in vitro susceptibility of Bacillus anthracis, Bacillus cereus and Bacillus thuringiensis against ertapenem, imipenem, cefoxitin, penicillin, tetracycline, and ciprofloxacin, MICs (minimum inhibitory concentrations) will be determined using E-test strips for all drugs. Bacillus cereus and Bacillus thuringiensis were also tested against norfloxacin, which was tested using the disk diffusion method.

Bacillus anthracis, and Bacillus cereus are deposited with the American Type Culture Collection, 10801 University Blvd., Manassas, Va. 20110-2209 as ATCC numbers 14578 and 6464, respectively. Bacillus. thuringiensis was deposited with the ATCC as ATCC# PTA 3863.

The assays used to test these compounds were performed essentially as described in National Committee for Clinical Laboratory Standards. Methods for Dilution Antimicrobial Susceptibility Tests for Bacteria that Grow Aerobically, Wayne, Pa., Approved Standard-Fifth Edition. M7-A5 Vol. 20 No.2. These are standard antibiotic susceptibility testing methods. Regarding safety, Biosafety in Microbiological and Biomedical Laboratories 4^{th} ed., US Dept of Health and Human Services; CDC and NIH, (1999) was consulted.

All work on the Bacillus anthracis, Bacillus cereus and Bacillus thuringiensis samples were performed in a biosafety cabinet Type II wearing gloves, a disposable gown, safety glasses, and a disposable respiratory mask (N-95). The biosafety cabinet was cleaned with 10% bleach before and after the work was performed. Any loops, swabs, pipettes, etc. used in the cabinet were disposed of into a container containing 10% bleach. The UV light was also on at all times when the cabinet was not in use. The room in which all work was performed had a combination lock on the door.

### EXAMPLE 1

In Vitro Susceptibility of Bacillus anthracis

Bacillus anthracis is known to be susceptible to penicillin, gentamicin, erythromycin, and chloramphenicol, people have also responded well to ciprofloxacin and doxycycline. Bacillus anthracis has now been confirmed to be susceptible to ertapenem and imipenem as shown below.

Materials and Methods

Blood agar plates (BBL-BAP) lot # 122532

Mueller Hinton agar plates—large (MH) (BBL) lot # 1228331

Trypticase soy broth (TS broth) (BBL) lot # 1254246

Thioglycollate broth (Thio) (BBL) lot # 221742

E-test strips:

Ertapenem—lot # BB-1710

Imipenem—lot #BA-3178

Cefoxitin—lot # BB-1364

Penicillin—lot # BA-2185

Tetracycline—lot # B82875

Ciprofloxacin—lot # B83457

Sterile swabs

Sterile loops

Biological safety cabinet Type II

Organisms—Bacillus anthracis ATCC 14578

Disposable lab coat

Gloves

Respiratory mask (N-95)

A sample Bacillus anthracis was subcultured onto a blood agar plate (BAP) using a sterile disposable loop and streaked for isolation. Approximately 0.1 mL of the, sample was also added to a trypticase soy broth (TS) broth and a thioglycollate broth using a sterile disposable pipette. The BAP was wrapped with parafilm and all three items (BAP, TSB, Thio) were placed in the 35° C. incubator. The organism was grown overnight @ 35° C. on blood agar plate (BAP), in TS broth, and in Thioglycollate broth. Pure and viable colonies of B. anthracis were observed, which were subcultured onto a new BAP and incubated @ 35° C. overnight. The TS broth and the Thio broth were cloudy and a 10% bleach solution was added to the tubes to decontaminate them. The tubes and the original BAP with growth (wrapped with parafilm) were discarded in the biosafety bag in the biosafety cabinet. Pure isolated colonies of B. anthracis were observed. With a sterile, disposable swab, a 0.5 McFarland was made in TS broth. The broth was vortexed, a new swab was moistened with the 0.5 McFarland suspension and 2 MH plates were swabbed in three directions. After drying (about 10 minutes), three E-test strips were placed on each MH plate for a total of six E-test strips (E-test strips were ertapenem, imipenem, penicillin, tetracycline, cefoxtin or ciprofloxacin). All plates were parafilmed and incubated at 35° C. overnight and the E-test MICs were recorded for each zone the next day according to manufacturer's instructions. The TS broth was decontaminated by adding 10% bleach solution to the tube. All plates and tubes containing B. anthracis were sealed with autoclave tape in a biohazard bag while in the biosafety cabinet. All lab coats and masks were sealed in another bag too. The two biohazard bags as well as the 10% bleach filled container with loops, swabs, and pipettes were autoclaved for 30 minuted @ 121° C. under 15 lbs. pressure. After autoclaving, the decontaminated goods were placed in a biohazard drum to be appropriately discarded.

Results—The MICs of B. anthracis vs 6 antibiotics are in the chart below.

| | Ertapenem | Imipenem | Cefoxitin | Penicillin | Tetracycline | Ciprofloxacin |
|---|---|---|---|---|---|---|
| | MIC | MIC | MIC | MIC | MIC | MIC |
| B. anthracis | 0.032 | 0.047 | 6 | 0.047 | 0.064 | 0.064 |
| ATCC 14578 | | | | | | |

### EXAMPLE 2

In Vitro Susceptibility of Bacillus cereus and B. thuringiensis

Bacillus cereus and Bacillus thuringiensis are known to produce a broad-spectrum β-lactamase and are therefore resistant to penicillin, ampicillin, and the cephalosporins. However, they are not resistant to imipenem, ciprofloxacin and tetracycline. Helgason et al., Applied and Environmental Microbiology 2000 66(6) pgs. 2627-2630 suggest that Bacillus anthracis, Bacillus cereus and Bacillus thuringiensis are one species based on genectic evidence. See also Afaro et al., Retina 1996; 16(4): 317-323.

Materials and Methods

Blood agar plates (BBL-TSA II) lot # 122532

Mueller Hinton agar plates—large (MH) (BBL) lot # 1228331

Mueller Hinton agar plates—small (MH) (BBL) lot # 125334

Trypticase soy broth (BBL) lot # 1254246

E-test strips:

Ertapenem—lot # BR-1677

Imipenem—lot #BA-3178

Cefoxitin—lot # BB-1364

Penicillin—lot # BA-2155

Tetracycline—lot # B82875

Ciprofloxacin—lot # B83457

Norfloxacin disks (BBL) lot # 1006-809624

Sterile swabs

Sterile loops

Turbidity meter (Dade/Behring)

Biological safety cabinet Type II

Organisms—8 Bacillus cereus and 2 Bacillus thuringiensis

Briefly, organisms were grown overnight @ 35° C. on blood agar plates (BAP) from either lyophilized MB isolates (MB refers to Merck Culture Collection as indicated below) or frozen CL#'s samples (CL refers to Clinical Culture Collection. After incubation, plates were checked for purity and viability and subcultured again to a BAP and incubated overnight @ 35° C. The next day, a 0.5 McFarland (standard term used for making a standardized inoculum through dilutions for antibiotic susceptibility testing; see the National Committee for Clinical Laboratory Standards. Methods for Dilution Antimicrobial Susceptibility Tests for Bacteria that Grow Aerobically, Wayne, Pa., Approved Standard-Fifth Edition. M7-A5 Vol. 20 No.2) was made in TS broth with each organism; each organism was then swabbed onto a large Mueller Hinton plate and a small Mueller Hinton plate. After 15 minutes, a norfloxacin disk was placed on each small MH plate; E-test strips of ertapenem (ERT), imipenem (IP), penicillin (PM), tetracycline (TC), cefoxitin (FX), and ciprofloxacin (CL) were placed on each large MH plate. The plates were incubated overnight @ 35° C. and the zones were recorded the next day according to manufacturer's instructions (AB Biodisk, Etest technical guide no.3 May 1993). See also Essential Procedures for Clinical Microbiology. Ed. Henry Isenberg. ASM Press, Washington DC 1998 Chap.5.8 E-test. Norfloxacin is abbreviated as (NX).

Results: The MICs of Bacillus cereus and Bacillus thuringiensis vs 7
antibiotics are in the chart below
Bacillus species E-Test MICs (except Norfloxacin =
Disk Diffusion)

| | | Organism | (PM) | (FX) | (TC) | (CL) | (IP) | (ERT) | (NX) |
|---|---|---|---|---|---|---|---|---|---|
| 1 | MB | B. thuringiensis | >32 | 24 | 1 | 0.19 | 0.75 ® | 0.25 | 24 mm |
| | 1074 | | | | | | | | |
| 2 | MB | B. cereus | >32 | 6 | 1 | 0.19 | 0.094 | 0.064 | 22 mm |
| | 5423 | | | | | | | | |
| 3 | MB | B. cereus | >32 | >256 | 24 | 0.19 | 1 ® | 0.094 | 22 mm |
| | 19 | | | | | | | | |
| 4 | MB | B. cereus | >32 | 64 | lab | 0.25 | 0.094 | 0.064 | 23 mm |
| | 871 | | | | accident | | | | |
| 5 | MB | B. cereus | >32 | 64 | 64 | 0.19 | 0.094 | 0.064 | 22 mm |
| | 3298 | | | | | | | | |
| 6 | MB | B. | >32 | 64 | 0.25 | 0.064 | 2 ® | 0.094 | 27 mm |
| | 1139 | thuringiensis | | | | | | | |
| 7 | CL | B. cereus | >32 | >256 | 0.5 | 0.19 | 4 ® | 0.064 | 23 mm |
| | 1940 | | | | | | | | |
| 8 | MB | B. cereus | >32 | 16 | 12 | 0.19 | 0.094 | 0.094 | 21 mm |
| | 5435 | | | | | | | | |
| 9 | CL | B. cereus | >32 | 16 | 0.38 | 0.064 | 0.064 | 0.094 | 25 mm |
| | 1938 | | | | | | | | |
| 10 | CL | B.cereus | >32 | 24 | 0.5 | 0.19 | 1 ® | 0.064 | 25 mm |
| | 1939 | | | | | | | | |

® = a few colonies grew at this MIC
Lab accident -E-test strip placed upside down

What is claimed is:

1. A method of inhibiting growth of Bacillus anthracis bacterial strains or homologs thereof in a mammal comprising administration to a patient in need thereof a therapeutically effective amount of a carbapenem compound.

2. A method of inhibiting growth of Bacillus anthracis bacterial strains or homologs thereof in a mammal comprising administration to a patient in need thereof a therapeutically effective amount of a compound of structural formula I: 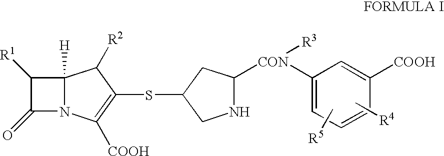
or a pharmaceutically acceptable salt, enantiomer, diastereomer or in vivo hydrolysable ester or mixture thereof:
wherein,
R¹ represents 1-hydroxyethyl, 1-fluoroethyl or hydroxymethyl;
R² and R³ independently represent hydrogen or C₁₋₄ alkyl;
R⁴ and R⁵ are the same or different and are selected from hydrogen, halo, cyano, C₁₋₄ alkyl, nitro, hydroxy, carboxy, C₁₋₄ alkoxy, C₁₋₄ alkoxycarbonyl, aminosulphonyl, C₁₋₄ alkylaminosulphonyl, di-C₁₋₄ alkylaminosulphonyl, carbamoyl, C₁₋₄ alkylcarbamoyl, di-C₁₋₄ alkylcarbamoyl, trifluoromethyl, sulphonic acid, amino, C₁₋₄ alkylamino, di-C₁₋₄ alkylamino, C₁₋₄ alkanoylamino, C₁₋₄ alkanoyl(N—C₁₋₄ alkyl)amino, C₁₋₄ alkanesulphonamido and C₁₋₄ alkylS(O)ₙ— wherein n is 0-2; and with the proviso that there is no hydroxy or carboxy substituent in a position ortho to the link to —NR³—.

3. The method according to claim 2 wherein R¹ is 1-hydroxyethyl, R² is hyrogen or methyl, R³ is hydrogen, and R⁴ and R⁵ are the same or different and are selected from hydrogen, fluoro, chloro, hydroxy, carboxy, cyano, nitro, methyl, ethyll, methoxy, ethoxy, methoxycarbonyl, carbamoyl, methylcarbamoyl, dimethylcarbamoyl, trifluoromethyl, sulphonic acid, methylsulphinyl, methylsulphonyl, methanesulphonamido or acetamido.

4. The method according to claim 3 wherein R⁴ is hydrogen, carboxy, fluoro, chloro, methyl, methoxy, cyano, sulphonic acid or methoxycarbonyl and R⁵ is hydrogen.

5. A method of inhibiting growth of Bacillus anthracis bacterial strains or homologs thereof in a mammal comprising administration to a patient in need thereof a therapeutically effective amount of a compound selected from the group consisting of:
(1R,5S,6S,8R,2′S,4′S)-2-(2-(3-carboxy-5-hydroxyphenyl-carbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-4-chlorophenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-6-chlorophenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxyphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-6-methanesulphonylphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxyfluorophenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-6-fluorophenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-2,4-difluorophenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3,4-dicarboxyphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-4-hydroxyphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3,5-dicarboxyphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(2-carbamoyl-3-carboxyphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-4-carbamoylphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-5-carbamoylphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-5-acetamidophenylcarbamoyl)pyrrolidin-4 ylthio)-6-(1-hydroxyethyl)-1-methylcarbapenem-3-carboxylic acid;
(1R,5S,6S,8R,2′S,4′S)-2-(2-(3-carboxy-4-acetamidophenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-5-methylsulphonamidophenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-5-sulphophenylcarbamoyl)pyrrolidin-4-ylthio)-6-(1-hydroxyethyl)-1-methylcarbapenem-3-carboxylic acid;
(1R,5,6S,8R,2′S,4′S)-2-(2-(3-carboxy-6-carbamoylphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-2-dimethylaminocarbonylphenylcarbamoyl)-pyrrolidin-4-ylthio)-6-(1-hydroxyethyl)-1-methylcarbapenem-3-carboxylic acid;
(1R,5S,6S,8R,2′S,4′S)-2-(2-(3-carboxyphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxymethylphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-5-methylphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-6-methylphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-2-methoxyphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxymethoxyphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-5-methoxyphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-6-methoxyphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-4,6-dimethoxyphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-5-methoxycarbonylphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-5-cyanophenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-5-trifluoromethylphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-4,6-difluorophenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-6-methylsulphinylphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-5-methylsulphonylphenylcarbamoyl)pyrrolidin-4-ylthio)-6-(1-hydroxyethyl)-1-methylcarbapenem-3-carboxylic acid;
(1R,5,6S,8R,2′S,4′S)-2-(2-(3-carboxy-5-fluorophenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-6-cyanophenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-N′-methylphenylcarbamoyl)pyrrolidin-4 ylthio)-6-(1-hydroxyethyl)-1-methylcarbapenem-3-carboxylic acid; and
pharmaceutically acceptable salts, enantiomers, diastereomers or in vivo hydrolysable esters or mixtures thereof.

6. A method according to claim 5 wherein the compound is:
(1R,5S,6S,8R,2′S,4′S)-2-(2-(3-carboxy-5-methylphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-5-methoxyphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-6-methoxyphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-5-methoxycarbonylphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-5-cyanophenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-6-chlorophenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxyphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxyfluorophenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-6-fluorophenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-dicarboxyphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3,5-dicarboxyphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-5-sulphophenylcarbamoyl)pyrrolidin-4 ylthio)-6-(1-hydroxyethyl)-1-methylcarbapenem-3-carboxylic acid;
or pharmaceutically acceptable salts, enantiomers, diastereomers or in vivo hydrolysable esters or mixtures thereof.

7. A method of inhibiting growth of Bacillus anthracis bacterial strains or homologs thereof in a mammal comprising administration to a patient in need thereof a therapeutically effective amount of (1R,5S,6S,8R,2′S,4′S)-2-(2-(3-carboxyphenylcarbamoyl)pyrrolidin-4-ylthio)-6-(1-hydroxyethyl)-1-methylcarbapenem-3-carboxylic acid in the form of a monosodium salt, disodium salt or mixture thereof.

8. A method of treating anthrax infection in a mammal comprising administration to a patient in need thereof a therapeutically effective amount of of a compound of structural formula I: 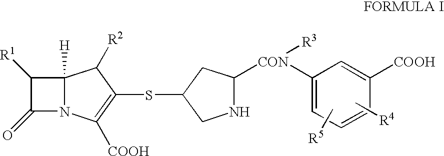
or a pharmaceutically acceptable salt, enantiomer, diastereomer or in vivo hydrolysable ester or mixture thereof:
wherein,
R¹ represents 1-hydroxyethyl, 1-fluoroethyl or hydroxymethyl;
R² and R³ independently represent hydrogen or C₁₋₄ alkyl;
R⁴ and R⁵ are the same or different and are selected from hydrogen, halo, cyano, C₁₋₄ alkyl, nitro, hydroxy, carboxy, C₁₋₄ alkoxy, C₁₋₄ alkoxycarbonyl, aminosulphonyl, C₁₋₄ alkylaminosulphonyl, di-C₁₋₄ alkylaminosulphonyl, carbamoyl, C₁₋₄ alkylcarbamoyl, di-C₁₋₄ alkylcarbamoyl, trifluoromethyl, sulphonic acid, amino, C₁₋₄ alkylamino, di-C₁₋₄ alkylamino, C₁₋₄ alkanoylamino, C₁₋₄ alkanoyl(N—C₁₋₄ alkyl)amino, C₁₋₄ alkanesulphonamido and C₁₋₄ alkylS(O)ₙ— wherein n is 0-2:
With the proviso tht there is no hydroxy or carboxy substituent in a position ortho to the link to —NR³—.

9. A method according to claim 8 wherein the compound is selected from the group consisting of:
(1R,5S,6S,8R,2′S,4′S)-2-(2-(3-carboxy-5-hydroxyphenyl-carbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-4-chlorophenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-6-chlorophenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxyphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-6-methanesulphonylphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-4-fluorophenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-6-fluorophenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-2,4-difluorophenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3,4-dicarboxyphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-4-hydroxyphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3,5-dicarboxyphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(2-carbamoyl-3-carboxyphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-4-carbamoylphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-5-carbamoylphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-5-acetamidophenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxyacetamidophenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-5-methylsulphonamidophenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-5-sulphophenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-6-carbamoylphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-2-dimethylaminocarbonylphenylcarbamoyl)-pyrrolidin-4-ylthio)-6-(1-hydroxyethyl)-1-methylcarbapenem-3-carboxylic acid;
(1R,5S,6S,8R,2′S,4′S)-2-(2-(3-carboxyphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-4-methylphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-5-methylphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-6-methylphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-2-methoxyphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-4-methoxyphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-5-methoxyphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-6-methoxyphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-4,6-dimethoxyphenylcarbamoyl)pyrrolidin-4 ylthio)-6-(1-hydroxyethyl)-1-methylcarbapenem-3-carboxylic acid;
(1R,5S,6S,8R,2′S,4′S)-2-(2-(3-carboxy-5-methoxycarbonylphenylcarbamoyl)pyrrolidin-4-ylthio)-6-(1-hydroxyethyl)-1-methylcarbapenem-3-carboxylic acid;
(1R,5S,6S,5R,2′S,4′S)-2-(2-(3-carboxy-5-cyanophenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-5-trifluoromethylphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-4,6-difluorophenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-6-methylsulphinylphenylcarbamoyl)pyrrolidinylthio)-6-(1-hydroxyethyl)-1-methylcarbapenem-3-carboxylic acid;
(1R,5S,6S,8R,2′S,4′S)-2-(2-(3-carboxy-5-methylsulphonylphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-5-fluorophenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-6-cyanophenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-N′-methylphenylcarbamoyl)pyrrolidin-4-ylthio)-6-(1-hydroxyethyl)-1-methylcarbapenem-3-carboxylic acid; and
pharmaceutically acceptable salts, enantiomers, diastereomers or in vivo hydrolysable esters or mixtures thereof.

10. A method according to claim 9 wherein the compounds is:
(1R,5S,6S,8R,2′S,4′S)-2-(2-(3-carboxy-5-methylphenylcarbamoyl)pyrrolidin-4 ylthio)-6-(1-hydroxyethyl)-1-methylcarbapenem-3-carboxylic acid;
(1R,5S,6S,8R,2′S,4′S)-2-(2-(3-carboxy-5-methoxyphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-6-methoxyphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-5-methoxycarbonylphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-5-cyanophenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-6-chlorophenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxyphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-4-fluorophenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-6-fluorophenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3,4-dicarboxyphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3,5-dicarboxyphenylcarbamoyl)pyrrolidin-4-ylthio)-6-(1-hydroxyethyl)-1-methylcarbapenem-3-carboxylic acid;
(1R,5S,6S,8R,2′S,4′S)-2-(2-(3-carboxy-5-sulphophenylcarbamoyl)pyrrolidin-4-ylthio)-6-(1-hydroxyethyl)-1-methylcarbapenem-3-carboxylic acid;
or pharmaceutically acceptable salts, enantiomers, diastereomers or in vivo hydrolysable esters or mixtures thereof.

11. A method of treating anthrax infection in a mammal comprising administration to a patient in need thereof a therapeutically effective amount of (1R,5S,6S,8R,2′S,4′S)-2-(2-(3-carboxyphenylcarbamoyl)pyrrolidin-4-ylthio)-6-(1-hydroxyethyl)-1-methylcarbapenem-3-carboxylic acid in the form of a monosodium salt, disodium salt or mixture thereof.

12. A method of inhibiting growth of Bacillus anthracis bacterial strain in a mammal comprising administration to a patient in need thereof a therapeutically effective amount of imipenem.

13. A method according to claim 12 wherein imipenem is combined with cilastatin.

14. A method according to claim 13 wherein the combination of imipenem and cilastatin is administered as PRIMAXIN®.

15. A method of treating anthrax infection in a mammal comprising administration to a patient in need thereof a therapeutically effective amount of imipenem.

16. A method according to claim 15 wherein imipenem is combined with cilastatin.

17. A method according to claim 16 wherein the combination of imipenem and cilastatin is administered as PRIMAXIN®.

18. A method according to claim 1 wherein the Bacillus antracis homologs include Bacillus cereus and Bacillus thuringiensis.
